(19) United States
(12) Patent Application Publication
(10) Pub. No.: US 2004/0259949 A1
(43) Pub. Date: Dec. 23, 2004

(54) Method

(76) Inventors: Jo Klaveness, Oslo (NO); Vidar Hansson, Oslo (NO)

Correspondence Address: KENYON & KENYON, ONE BROADWAY, NEW YORK, NY, 10004, US

(21) Appl. No.: 10/484,743

(22) PCT Filed: Jul. 25, 2002
(86) PCT No.: PCT/GB02/03386

(30) Foreign Application Priority Data
Jul. 26, 2001 (GB) 0118251.8

(51) Int. Cl. A61K 31/195

(52) U.S. Cl. 514/561

(57) ABSTRACT

The method relates to the use of a photosensitizer elected from 5-aminolevulinic acid (5-ALA) and 5-ALA derivatives, and pharmaceutically acceptable salts thereof, in the manufacture of a medicament for use in treating a wound. Examples of wounds which may be treated in accordance with the invention include those resulting from non-physiological processes, e.g. from surgery or from physical injury, abrasions, lacerations, and wounds arising from a thermal injury (e.g. a burn or a wound arising from any cryo-based treatment). Ulcers, e.g. leg ulcers, venous ulcer % and gastric ulcers, may also be successfully treated in accordance with the methods of the invention.

This invention relates to the treatment of wounds, and in particular to the use of 5-aminolevulinic acid (5-ALA) and 5-ALA derivatives in a method of accelerating wound healing.

In general, wound healing in healthy mammals proceeds quickly and with relatively few problems. However, the rate of the healing process is dependent upon several factors, including the nature of the wound (i.e. cause and size of the wound), the blood supply to the healing area, the presence and nature of any microorganisms and the general status of the patient (i.e. age, general health and dependence on any other drugs which may cause normal healing processes to be impaired or suppressed). Thus, in some cases, wound healing is delayed or impaired resulting in chronic or sub-chronic wounds which may take several months to heal. In severe cases, these may never fully heal. Chronic wounds can often result in complications and significant medical problems for the patient.

Typical problematic wounds (i.e. those which may be considered chronic or sub-chronic) include those arising from thermal injuries (including burns and wounds from freezing or cryo-based treatments), leg ulcers (including diabetic ulcers, e.g. neuropathic diabetic foot ulcers) and other chronic or sub-chronic ulcers (e.g. venous ulcers). Other types of ulcers which can cause problems during healing are those present in the gastrointestinal system, e.g. gastric ulcers.

One of the most serious problems associated with chronic or sub-chronic wounds is the possibility of bacterial infection, especially infections arising from Gram-negative anaerobic organisms. Infections in such wounds may, for example, be caused by Staphylococcus aureus, Pseudomonas and Proteus species. These infections can be very difficult to treat, especially in those cases where the bacteria are resistant to conventional antibiotics.

Conventionally, wounds may be treated using a combination of any of the following: different types of bandages, compressions and other wound dressings, local antiseptics, saline dressings, silver salts of antibacterial sulphonamides, topical and systemic antibiotics, benzoyl peroxide, zinc salts, vasodilators and skin grafting. For recent reviews on wound/ulcer healing see for example: S. Watanabe et al. in J. Gastroenterol. (2000) 35 Suppl. 12: 65-8, A. S. Rosemurgy et al. in Obes. Surg. (1991) 1: 145-149, I. Brook et al. in Pediatr. Neurosurg. (2000) 32: 20-23, C. E. Hallett et al. in J. Adv. Nurs. (2000) 31: 783-93, S. A. Kudravi et al. in In Vivo (2000) 14: 83-92, T. T. Phan et al. in Ann. Acad. Med. Singapore (2000) 29: 27-36, K. Takanagi et al. in Clin. Perform. Qual. Health Care (1999) 7: 70-73, A. Sheffet et al. in Ostomy Wound Manage (1999) 46: 28-33, 36-40, 42-44, S. Cerovac et al. in Burns (2000) 26: 251-259, H. J. Klasen in Burns (2000) 26: 207-22, A. K. Deodhar et al. in J. Postgrad. Med. (1997) 43: 52-56, C. Hernandez-Cueto et al. in Am. J. Forensic Med. Pathol. (2000) 21: 21-31, T. B. Burns et al. in Am. Fam. Physician (2000) 61: 1383-8, B. C. Ohanaka et al in East Afr. Med. J. (1999) 76, 687-9, L. Staiano-Cioco et al. in Ostomy Wound Manage (2000) 46 (1A Suppl), 85S-95S, P. D. Thomson in Ostomy Wound Manage (2000) 46 (1A Suppl) 77S-84S, M. Benbow in Community Nurse (1999) δ: 47-8 & 50, and M. Kiernan in Community Nurse (1999) δ: 59-60.

To date, several methods have been proposed to increase the rate of healing of wounds. Such methods include photodynamic therapy (PDT) using known photosensitizing agents. However, such methods have so far enjoyed limited success.

Photodynamic therapy (PDT) is a relatively new technique used in the treatment of various abnormalities or disorders of the skin or other epithelial organs or mucosa, especially cancer or pre-cancerous lesions, as well as certain non-malignant lesions such as psoriasis. PDT involves the administration of photosensitizing agents followed by exposure to photoactivating light in order to activate the photosensitizing agents and convert them into cytotoxic form resulting in the destruction of cells and thus treatment of the disease. Several photosensitizing agents are known and described in the literature, for example various porphyrins psorealens, chlorins, phthalocyanines and 5-aminolevulinic acid (5-ALA) derivatives.

Although PDT has focused on treatment of cancer and pre-cancerous stages there are some reports relating to PDT and wound healing. For example, U.S. Pat. No. 5,913,884 (The General Hospital Corporation) describes a method for modulating the healing of a wound in a mammal by administering an effective amount of a photosensitizer targeted to macrophages by conjugation to a targeting moiety. The targeting moiety conjugated to the photosensitizer may be selected from proteins, polypeptides and microparticles. A poly-1-lysine chlorin-e6 (ce6) conjugate is found to increase wound breaking strength in mice following PDT. However, no results are given for other photosensitizers.

More recent studies carried out by others clearly indicate that the wound healing effect seen when using a poly-1-lysine chlorin-e6 (ce6) conjugate in PDT is not observed when using other photosensitizing agents.

For example, Parekh et al. (Lasers Surg. Med. (1999) 24: 375-81) have studied the effect of two porphyrin-based photodynamically active agents. BDP-MA and CASP, on wound healing in rats. Their conclusion was that PDT did not influence skin wound healing in the rat model. This finding is confirmed by others. For example, studies carried out by A. Kübler et al. (Laser Surg. Med. (1996) 18: 397-405) have shown that PDT using Porphyrin results in delayed wound healing. M. J. Berlmont et al. (Laryngoscope (1999) 109: 886-90) also report delay of wound healing using PDT.

More recently, R. Haddad et al. (J. Gastroenterology (1999) 3: 602-6) reported the effect of photodynamic therapy on normal fibroblasts in colon anastomotic healing in mice using 5-ALA. It was concluded that PDT has no effect on viability of normal human fibroblasts and no significant impairment in healing of colonic anastomosis.

Therefore, at the present time it is generally accepted that well known photosensitizing agents have little or no effect on photodynamic wound healing.

A need still exists for alternative methods to increase the speed of healing of wounds, in particular chronic and sub-chronic wounds. Despite the negative results in the literature in relation to wound healing using known photosensitizers, we have now surprisingly found that 5-ALA and 5-ALA derivatives can be used clinically in photodynamic wound healing.

Thus, viewed from one aspect the invention provides the use of 5-ALA or a derivative or pharmaceutically acceptable salt thereof, in the manufacture of a medicament for use in treating wounds, e.g. chronic or sub-chronic wounds, in particular for use in a method of accelerating healing of wounds.

In a further aspect the invention provides a method of treatment of the human or non-human animal body to accelerate wound healing, said method comprising administering to a wound site in said body a photosensitizer selected from 5-ALA, a 5-ALA derivative and pharmaceutically acceptable salts thereof, and photoactivating said photosensitizer at the wound site.

In particular, the invention provides a method of treatment of the human or non-human animal body to accelerate wound healing, said method comprising the following steps:

(a) administering to a wound site in said body a photosensitizer selected from 5-ALA, 5-ALA derivatives and pharmaceutically acceptable salts thereof;

(b) if required, waiting for a time period necessary for the photosensitizer to achieve an effective tissue concentration at the wound site; and

(c) photoactivating the photosensitizer at the wound site.

The use of 5-ALA (5-amino-4-oxo-pentanoic acid, otherwise known as 5-aminolevulinic acid) and 5-ALA derivatives in PDT is well known in the scientific and patent literature (see, for example, J. C. Kennedy et al., J. Clin. Laser Med. Surg. (1996) 14: 289-304, US-A-5,079,262, U.S. Pat. No. 5,211,938, U.S. Pat. No. 5,234,940, U.S. Pat. No. 5,422,093, U.S. Pat. No. 6,034,267, WO91/01727 and WO96/28412, the contents of which are incorporated herein by reference). All such compounds and their pharmaceutially acceptable salts are suitable for use in the methods herein described.

The 5-ALA derivatives useful in accordance with the invention may be any derivative or analog of 5-ALA capable of forming protoporphyrin IX (PpIX) or any other photosensitizer (e.g. a PpIX derivative) in vivo. Typically, such derivatives will be a precursor of PpIX or of a PpIX derivative (e.g. a PpIX ester) in the biosynthetic pathway for haem and which are therefore capable of inducing an accumulation of PpIX at the site of the wound following administration in vivo. Suitable precursors of PpIX or PpIX derivatives include 5-ALA prodrugs which might be able to form 5-ALA in vivo as an intermediate in the biosynthesis of PpIX or which may be converted (e.g. enzymatically) to porphyrins without forming 5-ALA as an intermediate. 5-ALA and 5-ALA esters are among the preferred compounds for treatment of wounds in accordance with the invention.

Esters of 5-aminolevulinic acids and N-substituted derivatives thereof are preferred for use in the invention. Those compounds in which the 5-amino group is unsubstituted (i.e. the ALA esters) are particularly preferred. Such compounds are generally known and described in the literature (see, for example, WO96/28412 and WO02/10120 to PhotoCure ASA, the contents of which are incorporated herein by reference).

Esters of 5-aminolevulinic acids with optionally substituted alkanols, i.e. alkyl esters or substituted alkyl esters, are especially preferred for use in the invention. Examples of such compounds include those of general formula I:

R²₂N—CH₂COCH₂—CH₂CO—OR¹ (I)

(wherein

R¹ represents an optionally substituted straight-chained, branched or cyclic alkyl group; and each R² independently represents a hydrogen atom or an optionally substituted alkyl group, e.g. a group R¹) and pharmaceutically acceptable salts thereof.

As used herein, the term “alkyl”, unless stated otherwise, includes any long or short chain, cyclic, straight-chained or branched aliphatic saturated or unsaturated hydrocarbon group. The unsaturated alkyl groups may be mono- or polyunsaturated and include both alkenyl and alkynyl groups. Unless stated otherwise, such groups may contain up to 40 atoms. However, alkyl groups containing up to 30, preferably up to 10, particularly preferably up to 8, especially preferably up to 6, e.g. up to 4 carbon atoms are preferred.

The substituted alkyl R¹ and R² groups may be mono or poly-substituted. Suitable substituents may be selected from hydroxy, alkoxy, acyloxy, alkoxycarbonyloxy, amino, aryl, nitro, oxo, fluoro, —SR³, —NR³₂ and —PR³₂ groups, and each alkyl group may be optionally interrupted by one or more —O—, —NR³—, —S— or —PR³— groups, in which R³ is a hydrogen atom or a C₁₋₆ alkyl group).

Particularly preferred for use in the invention are those compounds of formula I in which R¹ either represents an unsubstituted alkyl group (e.g. C₁₋₆ alkyl) or an alkyl group (e.g. C₁₋₂ alkyl) substituted by an aryl group (e.g. phenyl) and/or each R² represents a hydrogen atom.

Especially preferred compounds of formula I include 1-methylpentyl ALA ester, p-isopropylbenzyl ALA ester, p-methylbenzyl ALA ester, benzyl ALA ester, 2-phenylethyl ALA ester, hexyl ALA ester, cyclohexyl ALA ester, 4-methylpentyl ALA ester, p-[trifluoromethyl]benzyl ALA ester, p-[t-butyl]benzyl ALA ester, p-nitrobenzyl ALA ester, 1-ethylbutyl ALA ester, 2-methylpentyl ALA ester, 4-phenyl butyl ALA ester, p-fluorobenzyl ALA ester, 3,3-dimethyl-1-butyl ALA ester, 2-fluorobenzyl ALA ester, 2,3,4,5,6-pentafluorobenzyl ALA ester, 4-chlorobenzyl ALA ester, 2-methoxyethyl ALA ester, 3-nitrobenzyl ALA ester, 3,4-[di-chloro]benzyl ALA ester, 3,6-dioxa-1-octyl ALA ester, 3-fluorobenzyl ALA ester, 3,6,9-trioxa-1-decyl ALA ester, 3-pyridinyl-methyl ALA ester, 4-diphenyl-methyl ALA ester, 4-methoxy-benzyl ALA ester, 2-methylbenzyl ALA ester, benzyl-5-[(1-acetyloxyethoxy)-carbonyl]amino levulinate, and 3-methylbenzyl ALA ester Most preferred for use in the method of the invention are 5-ALA, 5-ALA methyl ester, 5-ALA hexyl ester and 5-ALA benzyl ester.

The compounds for use according to the method of the invention may be in the form of a free amine and/or acid or in the form of a physiologically acceptable salt. Such salts preferably are acid addition salt with physiologically acceptable organic or inorganic acids. Suitable acids include, for example, hydrochloric, hydrobromic, sulphuric, phosphonic, acetic, lactic, citric, tartaric, succinic, maleic, fumaric, and ascorbic acids. Procedures for salt formation are conventional in the art.

In the method of the invention a single photosensitizer (i.e. 5-ALA or 5-ALA derivative) may be used alone in treating the wound. Alternatively, a combination of two or more, preferably two, photosensitizers may be used wherein at least one of the photosensitizers is 5-ALA, a derivative of 5-ALA or a pharmaceutically acceptable salt thereof.

Other photosensitizers which may be formulated with 5-ALA or a 5-ALA derivative or co-administered in accordance with the invention include:

Hematoporphyrin derivative (HpD);

Hematoporphyrins such as Photofrin® (Quadra Logic Technologies Inc., Vancouver, Canada) and Hematoporphyrin IX (HpIX);

Photosan III (Seehof Laboratorium GmbH, Seehof, Wesselburenerkoog, Germany);

Chlorins such as tetra(m-hydroxyphenyl)chlorins (m-THPC) and their bacteriochlorins (Scotia Pharmaceuticals Ltd, Surrey, UK), mono-L-aspartyl chlorin e6 (NPe6) (Nippon Petrochemical Co., CA, USA), chlorin e6 (Porphyrin Products Inc.), benzoporphyrins (Quadra Logic Technologies Inc., Vancouver, Canada) (e.g. benzoporphyrin derivative monoacid ring A, BPD-MA) and purpurines (PDT Pharmaceuticals Inc., CA, USA)(e.g. tin-ethyl etiopurpurin, SnET2);

phthalocyanines (e.g. zinc-(Quadra Logic Technologies Inc., Vancouver, Canada), some aluminium- or silicon phthalocyanines, which may be sulfonated, in particular sulfonated phthalocyanines such as aluminium phthalocyanine di-sulfonate (AlPcS₂ₐ) or aluminium phthalocyanine tetra-sulfonate (AlPcS₄));

porphycenes;

hypocrellins;

Protoporphyrin IX (PpIX);

Hematoporphyrin di-ethers;

Uroporphyrins;

Coproporphyrins;

Deuteroporphyrin; and

Polyhematoporphyrin (PHP), and precursors and derivatives thereof.

Preferably the second photosensitizer will be a Hematoporphyrin (e.g. Photofrin®), a chlorin (particularly m-THPC or chlorin e6) or a sulphonated phthalocyanine (particularly aluminium phthalocyanine di-sulfonate or aluminium phthalocyanine tetra-sulfonate).

In a further aspect the invention thus provides the use of a first photosensitizer selected from 5-ALA, 5-ALA derivatives and pharmaceutically acceptable salts thereof, together with a second photosensitizer in the manufacture of a medicament for use in treating wounds, e.g. chronic or sub-chronic wounds, in particular for use in a method of accelerating wound healing.

In a yet further aspect the invention provides the use of a first photosensitizer selected from 5-ALA, 5-ALA derivatives and pharmaceutically acceptable salts thereof, together with a second photosensitizer in the manufacture of medicaments for simultaneous, separate or sequential use in a method of treating wounds.

In a still further aspect the invention provides a kit or pack containing a first photosensitizer selected from 5-ALA, 5-ALA derivatives and pharmaceutically acceptable salts thereof, and separately a second photosensitizer for simultaneous, separate or sequential use in treating wounds, e.g. chronic or sub-chronic wounds.

As used herein, the term “wound” includes any disruption and/or loss of normal tissue continuity in an internal or external body surface of a human or non-human animal body, e.g. resulting from a non-physiological process such as surgery or physical injury. Treatment of a wound as described herein is not intended to encompass direct treatment of any build-up of abnormal cells within the body, e.g. a tumor.

Any wound in a human or non-human mammal, e.g. a human, especially problematic wounds such as chronic and sub-chronic wounds may be treated in accordance with the invention. Such wounds may result from surgery or physical injury, or may be associated with certain disease states (e.g. ulcers). The wound may be present on any external or internal body surface and may be penetrating or non-penetrating. Internal and external body surfaces which may be treated in accordance with the invention include the skin, the lining of the mouth, the pharynx, the esophagus, and the lining of the stomach and intestines. The method herein described is particularly beneficial in treating problematic wounds on the skin's surface. Examples of wounds which may be treated in accordance with the method of the invention include both superficial and non-superficial wounds, e.g. abrasions, lacerations, wounds arising from thermal injuries (e.g. burns and those arising from any cryo-based treatment), and any wound resulting from surgery.

Wounds to be treated in accordance with the methods herein described will preferably be non-infected or essentially clean wounds in which any microorganism(s), e.g. bacteria, which may be present will not prevent the wound from healing. Such wounds will, in general, be substantially free from (e.g. free from) any pathogenic microorganism(s). In particular, these can be expected to be substantially free from any infection of bacterial origin such as Staphylococcus aureus, Staphylococcus epidermidis, etc. Generally speaking, such wounds will be free from any opportunist infection.

Ulcers, such as leg ulcers, venous ulcers or those present in the gastrointestinal tract, e.g. gastric ulcers, may also be treated using the methods herein described. Such methods have been found to be particularly suitable for the treatment of neuropathic diabetic foot ulcers.

The compounds for use according to the invention can be formulated in conventional manner with one or more physiologically acceptable carriers or excipients according to techniques well known in the art.

Compositions may be administered locally at or near the wound site (e.g. topically or by injection) or systemically (e.g. orally or parenterally). The route of administration will depend on the size and nature of the wound to be treated, the location of the wound and the photosensitizer (or combination of photosensitizers) used. In cases where the size, nature and location of the wound permits local administration of the formulation, local administration is preferred (either to an internal or external body surface). Preferred formulations include gels, creams, ointments, sprays, lotions, salves, sticks, soaps, powders, pessaries, aerosols, drops, solutions and any other conventional pharmaceutical forms in the art.

Ointments, gels and creams may, for example, be formulated with an aqueous or oily base with the addition of suitable thickening and/or gelling agents. Lotions may be formulated with an aqueous or oily base and will, in general, also contain one or more emulsifying, dispersing, suspending, thickening or colouring agents. Powders may be formed with the aid of any suitable powder base. Drops and solutions may be formulated with an aqueous or non-aqueous base also comprising one or more dispersing, solubilising or suspending agents. Aerosol sprays are conveniently delivered from pressurised packs, with the use of a suitable propellant.

The composition may additionally include lubricating agents, wetting agents, emulsifying agents, suspending agents, preserving agents, sweetening agents, flavouring agents, adsorption enhancers, e.g. surface penetrating agents as mentioned below, and the like. The compositions for use in the method of the invention may be formulated so as to provide quick, sustained or delayed release of the active ingredient after administration to the patient by employing procedures well known in the art. Solubilizing and/or stabilizing agents may also be used, e.g. cyclodextrins (CD) α, β, γ and HP-β cyclodextrin. Compositions may be in any appropriate dosage form, for example as an emulsion or in liposomes, niosomes, microspheres, nanoparticles or the like. The compound for use in the invention may then be absorbed to, incorporated in or bound to these forms.

Typically, compositions for PDT wound healing will be in the form of a ready-to-use formulation such as a cream (for example Metvix® cream containing 5-ALA methyl ester at 20% (w/w)) or as a kit consisting of a two component system (e.g. containing two photosensitizing agents).

The pH in the final-formulation is preferably in the range 2.5 to 7.4. Acidic pH, for example pH 5, is preferred if the formulation is a ready-to-use formulation.

The concentration of the 5-ALA compounds described above in the final formulation for treatment of wounds will vary depending on several factors including the chemical nature of the compound, the chemical composition, mode of administration and nature of the wound to be treated. Generally, however, concentration ranges between 0.01 to 30% (w/w) are suitable. The most preferred concentrations for wound healing with local administration is in the range 0.02 to 25% (w/w), e.g. about 20% (w/w).

Topical administration to inaccessible sites may be achieved by techniques known in the art, e.g. by the use of catheters or other appropriate drug delivery systems.

After administration of the pharmaceutical formulation containing the photosensitizer(s), the site of the wound is exposed to light to achieve the desired photosensitizing effect. The length of time following administration, at which the light exposure takes place will depend on the nature of the composition, the condition to be treated and the form of administration. Generally, it is necessary that the photosensitizer should reach an effective tissue concentration at the site of the wound prior to photoactivation. This can generally take in the region of from 1 to 24 hours.

In a preferred treatment procedure, the photosensitizer(s) is/are applied to the wound followed by irradiation after a period of about 3 hours. If necessary, this procedure may be repeated, e.g. up to a further 3 times, at intervals of up to 14 days (e.g. 7-14 days). In those cases where this procedure does not lead to complete healing of the wound, an additional treatment may be performed several months later.

The irradiation will in general be applied at a dose level of 40 to 200 Joules/cm², for example at 100 Joules/cm².

The wavelength of light used for irradiation may be selected to achieve a more efficacious photosensitizing effect. The most effective light is light in the wavelength range 300-800 nm, typically 400-700 nm.

A further aspect of the invention thus provides a method of treating a wound in a mammal (e.g. a human), said method comprising administering to the site of the wound a composition as hereinbefore defined, and exposing said surface to light, preferably to light in the wavelength region 300-800 nm, for example 400-700 nm.

Methods for irradiation of different areas of the body, eg. by lamps or lasers, are well known in the art (see for example Van den Bergh, Chemistry in Britain, May 1986 p. 430-439). For inaccessible regions this may conveniently be achieved using optical fibres.

As hereinbefore described, the compounds for use in the invention may be formulated and/or administered with other photosensitizing agents, for example 5-ALA or another 5-ALA derivative, or a porphyrin derivative such as Photofrin®. Alternatively, these may be formulated and/or administered with other active components which are able to increase the photosensitizing effect and thus enhance wound healing. For example, chelating agents may beneficially be included and/or co-administered in order to enhance the accumulation of Pp; the chelation of iron by the chelating agent prevents its incorporation into Pp to form haem by the action of the enzyme ferrochelatase, thereby leading to a build-up of Pp. The photosensitizing effect is thus enhanced.

Suitable chelating agents include aminopolycarboxylic acids, including any of the chelants described in the literature for metal detoxification or for the chelation of paramagnetic metal ions in magnetic resonance imaging contrast agents. Particular mention may be made of EDTA, CDTA (cyclohexane diamine tetraacetic acid), DTPA and DOTA and well known derivatives/analogues thereof. EDTA and DTPA are particularly preferred. To achieve the iron-chelating effect, desferrioxamine and other siderophores may also be used, e.g. in conjunction with aminopolycarboxylic acid chelating agents such as EDTA.

Where present, the chelating agent may conveniently be used at a concentration of 0.05 to 20%, e.g. 0.1 to 10% (w/w).

Penetration enhancers may also have a beneficial effect in enhancing the photosensitizing effect of the compounds for use in the invention. Surface-penetration assisting agents, especially dialkylsuphoxides such as dimethylaulphoxide (DMSO), may therefore also be included in the compositions for use in the invention and/or co-administered. The surface-penetration assisting agent may be any of the skin-penetration assisting agents described in the pharmaceutical literature e.g. chelators (e.g. EDTA), surfactants (e.g. sodium dodecyl sulphate), non-surfactants, bile salts (e.g. sodium deoxycholate) and fatty acids (e.g. oleic acid). Examples of appropriate surface penetrating assisting agents include HPE-101 (available from Hisamitsu), DMSO and other dialkylsulphoxides, in particular n-decylmethyl-sulphoxide (NDMS), dimethylsulphacetamide, dimethylformamide (DMFA), dimethylacetamide, glycols, various pyrrolidone derivatives (Woodford et al., J. Toxicol. Cut. & Ocular Toxicology, 1986, 5: 167-177), and Azone® (Stoughton et. al., Drug Dpv. Ind. Pharm. 1983, 9: 725-744), or mixtures thereof. DMSO is, however, preferred due to its anti-histamine and anti-inflammatory activities and its stimulatory effect on the activity of the enzymes ALA-synthase and ALA-dehydrogenase (the enzymes which, respectively, form and condense ALA to porphobilinogen) thereby enhancing the formation of the active form, Pp.

The surface penetration agent may conveniently be provided in a concentration range of 0.2 to 50% (w/w), e.g. about 10% (w/w).

Viewed from a further aspect, the invention thus provides the use of 5-ALA, a 5-ALA derivative, or a pharmaceutically acceptable salt thereof, together with at least one surface-penetration assisting agent, and optionally one or more chelating agents, in the manufacture of a medicament or medicaments for use in the treatment of wounds, in particular chronic or sub-chronic wounds.

The compounds for use in the invention may additionally be used in combination with other non-photosensitizing agents to improve wound healing. Such agents include antiseptics and antibiotics, e.g. bacitracin. Although these may be present as part of the formulation, typically these will be used as a separate treatment to be administered simultaneously, separately or sequentially. Administration of any supplementary agent should be performed in terms of route, concentration and formulation, according to known methods for using these agents. These additional agents may be administered before, during or after PDT, depending on their function.

Viewed from a further aspect the invention thus provides a product or kit for use in a method of treating wounds comprising:

(a) a first container containing 5-ALA, a 5-ALA derivative or a pharmaceutically acceptable salt thereof; and

(b) a second container containing an antiseptic or an antibiotic.

Additional components of the kit may also be provided such as a second photosensitizing agent, a surface-penetrating agent or a chelating agent as herein described.

Depending on the nature of the wound to be treated, and the nature of any additional active agent or agents to be used in the method of the invention, this may be co-administered with the 5-ALA/5-ALA derivative, for example in a single composition, or this may be administered sequentially or separately. Typically, in those cases where a surface-penetration assisting agent is used, this will be administered in a separate step prior to administration of the compounds for use in the invention. When a surface-penetration assisting agent is used in pre-treatment this may be used at high concentrations, e.g. up to 100% (w/w). If such a pre-treatment step is employed, the photosensitizing agent may subsequently be administered up to several hours following pre-treatment, e.g. at an interval of 5-60 minutes following pre-treatment.

The invention will now be described in more detail by way of the following non-limiting Example:

### EXAMPLE

A 78 year old man with actinic keratosis (AL) (sun-damaged skin) on the head/skull developed erosions and wounds (size approx. 15×10 cm) following cryotherapy with liquid nitrogen (to freeze and kill abnormal cells). Standard wound treatment (vaseline compress, saline compresses) was unsuccessful in healing the wound.

After 3 months without healing, the wounded area was covered with a 20% 5-ALA methyl ester cream (Metvix® available from Photocure ASA, Oslo). Three hours later the area was exposed to light (420 nm) at a dose level of 5 J/cm². After only one PDT procedure and within 4 weeks, normal re-epithelisation of the area was observed (with the exception of a small area approx. 10×20 mm in size).

The remaining area was subjected to a second PDT procedure following further application of Metvix® (irradiation 3 hours after application with red light (570-670 nm) and 50 J/cm²). This resulted in complete healing of the remaining wound area within a further 4 weeks.

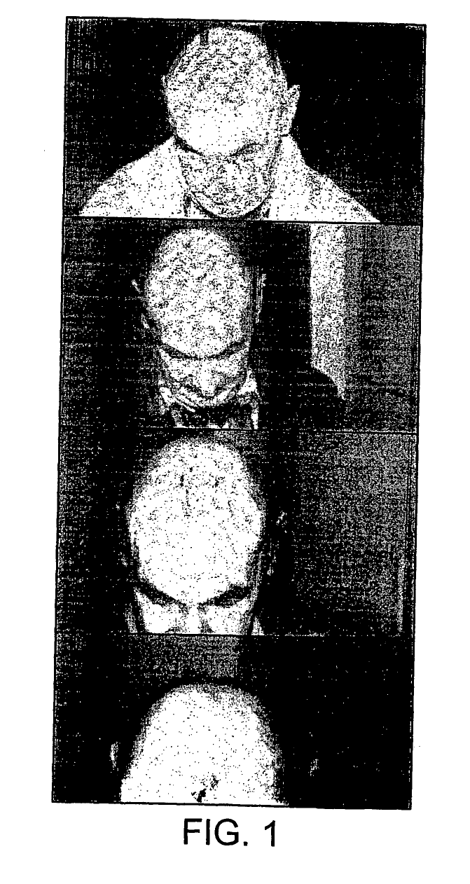

Attached FIG. 1 illustrates the wound healing process following treatment as outlined above.

1-21. (canceled).

22. A method for manufacturing a medicament for use in treating a wound arising from a thermal injury or a wound associated with an ulcer, said method comprising formulating a photosensitizer which is a derivative or analog of 5-aminolevulinic acid (5-ALA), or pharmaceutically acceptable salts thereof, with one or more physiologically acceptable carrier or excipient.

23. The method of claim 22 further comprising formulating the medicament with a surface-penetration assisting agent, a chelating agent, or a surface-penetration assisting agent and a chelating agent.

24. The method of claim 22, wherein said photosensitizer is a derivative or analog of 5-ALA capable of forming protoporphyrin 1× or a protoporphyrin IX derivative in vivo.

25. The method of claim 24, wherein said photosensitizer is an ester of 5-ALA or an N-substituted derivative thereof.

26. The method of claim 25, wherein said photosensitizer is a compound of general formula I:
R²₂N—CH₂COCH₂—CH₂CO—OR¹ (I)
wherein R¹ represents an optionally substituted straight-chained, branched or cyclic alkyl group; and each R² independently represents a hydrogen atom or an optionally substituted alkyl group, or a pharmaceutically acceptable salt thereof.

27. The method of claim 26, wherein in each R² the optionally substituted alkyl group is an R¹ group.

28. The method of claim 26, wherein in formula I, R¹ either represents an unsubstituted alkyl group or an alkyl group substituted by an aryl group and/or each R² represents a hydrogen atom.

29. The method of claim 28, wherein the unsubstituted alkyl group is a C₁₋₆ alkyl and the alkyl group substituted by the aryl group is a C₁₋₂ alkyl.

30. The method of claim 29, wherein the aryl group is phenyl.

31. The method of claim 26, wherein said compound is selected from the group consisting of 1-methylpentyl ALA ester, p-isopropylbenzyl ALA ester, p-methylbenzyl ALA ester, benzyl ALA ester, 2-phenylethyl ALA ester, hexyl ALA ester, cyclohexyl ALA ester, 4-methylbenzyl ALA ester, p-[tri-fluoromethyl]benzyl ALA ester, p-[t-butyl]benzyl ALA ester, p-nitrobenzyl ALA ester, 1-ethylbutyl ALA ester, 2-methylbenzyl ALA ester, 4-phenyl butyl ALA ester, p-fluorobenzyl ALA ester, 3,3-dimethyl-1-butyl ALA ester, 2-fluorobenzyl ALA ester, 2,3,4,5,6-pentafluorobenzyl ALA ester, 4-chlorobenzyl ALA ester, 2-methoxyethyl ALA ester, 3-nitrobenzyl ALA ester, 3,4-[di-chloro]benzyl ALA ester, 3,6-dioxa-1-octyl ALA ester, 3-fluorobenzyl ALA ester, 3,6,9-trioxa-1-decyl ALA ester, 3-pyridinyl-methyl ALA ester, 4-diphenyl-methyl ALA ester, 4-methoxy-benzyl ALA ester, 2-methylbenzyl ALA ester, benzyl-5-[(11-acetyloxyethoxy)-carbonyl]amino levulinate, and 3-methylbenzyl ALA ester, and pharmaceutically acceptable salts thereof.

32. The method of claim 25, wherein said photosensitizer is 5-ALA methyl ester, 5-ALA hexyl ester, 5-ALA benzyl ester, or pharmaceutically acceptable salts thereof.

33. The method of claim 22, wherein the thermal injury arises from a bum or from a cryo-based treatment.

34. The method of claim 22, wherein the thermal injury or the wound associated with an ulcer wound is substantially free from infection by a pathogenic microorganism.

35. The method of claim 22, said ulcer is a leg ulcer, venous ulcer or gastric ulcer.

36. A method of treatment of a human or non-human animal body to accelerate healing of a wound arising from a thermal injury or a wound associated with an ulcer, said method comprising administering to a wound site in said body a photosensitizer which is a derivative or analog of 5-aminolevulinic acid (5-ALA), or pharmaceutically acceptable salts thereof, formulated with one or more physiologically acceptable carrier or excipient, optionally in combination with a surface-penetration assisting agent and/or a chelating agent, and photoactivating said photosensitizer at the wound site.

37. The method of claim 36, wherein said photosensitizer is an ester of 5-ALA or an N-substituted derivative thereof.

38. The method of claim 37, wherein said photosensitizer is a compound of general formula I:
R²₂N—CH₂COCH₂—CH₂CO—OR¹ (I)
wherein R¹ represents an optionally substituted straight-chained, branched or cyclic alkyl group; and
each R² independently represents a hydrogen atom or an optionally substituted alkyl group, or a pharmaceutically acceptable salt thereof.

39. The method of claim 38, wherein in each R² the optionally substituted alkyl group is an R¹ group.

40. The method of claim 38, wherein in formula I, R¹ either represents an unsubstituted alkyl group or an alkyl group substituted by an aryl group and/or each R² represents a hydrogen atom.

41. The method of claim 38, wherein said compound is selected from the group consisting of 1-methylpentyl ALA ester, p-isopropylbenzyl ALA ester, p-methylbenzyl ALA ester, benzyl ALA ester, 2-phenylethyl ALA ester, hexyl ALA ester, cyclohexyl ALA ester, 4-methylbenzyl ALA ester, p-[tri-fluoromethyl]benzyl ALA ester, p-[t-butyl]benzyl ALA ester, p-nitrobenzyl ALA ester, 1-ethylbutyl ALA ester, 2-methylbenzyl ALA ester, 4-phenyl butyl ALA ester, p-fluorobenzyl ALA ester, 3,3-dimethyl-1-butyl ALA ester, 2-fluorobenzyl ALA ester, 2,3,4,5,6-pentafluorobenzyl ALA ester, 4-chlorobenzyl ALA ester, 2-methoxyethyl ALA ester, 3-nitrobenzyl ALA ester, 3,4-[di-chloro]benzyl ALA ester, 3,6-dioxa-1-octyl ALA ester, 3-fluorobenzyl ALA ester, 3,6,9-trioxa-1-decyl ALA ester, 3-pyridinyl-methyl ALA ester, 4-diphenyl-methyl ALA ester, 4-methoxy-benzyl ALA ester, 2-methylbenzyl ALA ester, benzyl-5-[(1-acetyloxyethoxy)-carbonyl]amino levulinate, and 3-methylbenzyl ALA ester, and pharmaceutically acceptable salts thereof.

42. The method of claim 37, wherein said photosensitizer is 5-ALA methyl ester, 5-ALA hexyl ester, 5-ALA benzyl ester, or pharmaceutically acceptable salts thereof.

43. The method of claim 36, wherein the step of photoactivating the photosensitizer is effected by exposing the wound site to light in the wavelength region 300-800 nm.

44. A method of treatment of a human or non-human animal body to accelerate healing of a wound arising from a thermal injury or a wound associated with an ulcer, said method comprising the following steps:
(a) administering to a wound site in said body a photosensitizer which is a derivative or analog of 5-aminolevulinic acid (5-ALA), or pharmaceutically acceptable salts thereof, formulated with one or more physiologically acceptable carrier or excipient, optionally in combination with a surface-penetration assisting agent and/or a chelating agent;
(b) if required, waiting for a time period necessary for the photosensitizer to achieve an effective tissue concentration at the wound site; and
(c) photoactivating the photosensitizer at the wound site.

45. The method of claim 44, wherein said photosensitizer is an ester of 5-ALA or an N-substituted derivative thereof.

46. The method of claim 45, wherein said photosensitizer is a compound of general formula I:
R²₂N—CH₂COCH₂—CH₂CO—OR¹ (I)
wherein R¹ represents an optionally substituted straight-chained, branched or cyclic alkyl group; and
each R² independently represents a hydrogen atom or an optionally substituted alkyl group, or a pharmaceutically acceptable salt thereof.

47. The method of claim 46, wherein in each R² the optionally substituted alkyl group is an R¹ group.

48. The method of claim 46, wherein in formula I, R¹ either represents an unsubstituted alkyl group or an alkyl group substituted by an aryl group and/or each R² represents a hydrogen atom.

49. The method of claim 46, wherein said compound is selected from the group consisting of 1-methylpentyl ALA ester, p-isopropylbenzyl ALA ester, p-methylbenzyl ALA ester, benzyl ALA ester, 2-phenylethyl ALA ester, hexyl ALA ester, cyclohexyl ALA ester, 4-methylbenzyl ALA ester, p-[tri-fluoromethyl]benzyl ALA ester, p-[t-butyl]benzyl ALA ester, p-nitrobenzyl ALA ester, 1-ethylbutyl ALA ester, 2-methylbenzyl ALA ester, 4-phenyl butyl ALA ester, p-fluorobenzyl ALA ester, 3,3-dimethyl-1-butyl ALA ester, 2-fluorobenzyl ALA ester, 2,3,4,5,6-pentafluorobenzyl ALA ester, 4-chlorobenzyl ALA ester, 2-methoxyethyl ALA ester, 3-nitrobenzyl ALA ester, 3,4-[di-chloro]benzyl ALA ester, 3,6-dioxa-1-octyl ALA ester, 3-fluorobenzyl ALA ester, 3,6,9-trioxa-1-decyl ALA ester, 3-pyridinyl-methyl ALA ester, 4-diphenyl-methyl ALA ester, 4-methoxy-benzyl ALA ester, 2-methylbenzyl ALA ester, benzyl-5-[(1-acetyloxyethoxy)-carbonyl]amino levulinate, and 3-methylbenzyl ALA ester, and pharmaceutically acceptable salts thereof.

50. The method of claim 45, wherein said photosensitizer is 5-ALA methyl ester, 5-ALA hexyl ester, 5-ALA benzyl ester, or pharmaceutically acceptable salts thereof.

51. The method of claim 44, wherein the step of photoactivating the photosensitizer is effected by exposing the wound site to light in the wavelength region 300-800 nm.

52. A method for manufacturing a medicament for use in treating a wound arising from a thermal injury or a wound associated with an ulcer, said method comprising formulating a first photosensitizer which is a derivative or analog of 5-aminolevulinic acid (5-ALA), or pharmaceutically acceptable salts thereof, and a second photosensitizer with one or more physiologically acceptable carrier or excipient.

53. The method of claim 52, wherein said first photosensitizer is an ester of 5-ALA or an N-substituted derivative thereof.

54. The method of claim 53, wherein said first photosensitizer is a compound of general formula I:
R²₂N—CH₂COCH₂—CH₂CO—OR¹ (I)
wherein R¹ represents an optionally substituted straight-chained, branched or cyclic alkyl group; and
each R² independently represents a hydrogen atom or an optionally substituted alkyl group, or a pharmaceutically acceptable salt thereof.

55. The method of claim 54, wherein in each R² the optionally substituted alkyl group is an R¹ group.

56. The method of claim 54, wherein in formula I, R¹ either represents an unsubstituted alkyl group or an alkyl group substituted by an aryl group and/or each R² represents a hydrogen atom.

57. The method of claim 54, wherein said compound is selected from the group consisting of 1-methylpentyl ALA ester, p-isopropylbenzyl ALA ester, p-methylbenzyl ALA ester, benzyl ALA ester, 2-phenylethyl ALA ester, hexyl ALA ester, cyclohexyl ALA ester, 4-methylbenzyl ALA ester, p-[tri-fluoromethyl]benzyl ALA ester, p-[t-butyl]benzyl ALA ester, p-nitrobenzyl ALA ester, 1-ethylbutyl ALA ester, 2-methylbenzyl ALA ester, 4-phenyl butyl ALA ester, p-fluorobenzyl ALA ester, 3,3-dimethyl-1-butyl ALA ester, 2-fluorobenzyl ALA ester, 2,3,4,5,6-pentafluorobenzyl ALA ester, 4-chlorobenzyl ALA ester, 2-methoxyethyl ALA ester, 3-nitrobenzyl ALA ester, 3,4-[di-chloro]benzyl ALA ester, 3,6-dioxa-1-octyl ALA ester, 3-fluorobenzyl ALA ester, 3,6,9-trioxa-1-decyl ALA ester, 3-pyridinyl-methyl ALA ester, 4-diphenyl-methyl ALA ester, 4-methoxy-benzyl ALA ester, 2-methylbenzyl ALA ester, benzyl-5-[(1-acetyloxyethoxy)-carbonyl]amino levulinate, and 3-methylbenzyl ALA ester, and pharmaceutically acceptable salts thereof.

58. The method of claim 53, wherein said first photosensitizer is 5-ALA methyl ester, 5-ALA hexyl ester, 5-ALA benzyl ester, or pharmaceutically acceptable salts thereof.

59. The method of claim 52, wherein said second photosensitizer is a hematoporphyrin, a chlorin, or a sulphonated phthalocyanine.

60. A method of treating a wound arising from a thermal injury or a wound associated with an ulcer, said method comprising administering to a wound site in said body a first photosensitizer which is a derivative or analog of 5-aminolevulinic acid (5-ALA), or pharmaceutically acceptable salts thereof, formulated with one or more physiologically acceptable carrier or excipient, optionally in combination with a surface-penetration assisting agent and/or a chelating agent; administering to the wound site in the body a second photosensitizer and photoactivating said first photosensitizer; wherein the first photosensitizer and second photosensitizer are administered simultaneously, separately or sequentially.

61. The method of claim 60, wherein said first photosensitizer is an ester of 5-ALA or an N-substituted derivative thereof.

62. The method of claim 61, wherein said first photosensitizer is a compound of general formula I:
R²₂N—CH₂COCH₂—CH₂CO—OR¹ (I)
wherein R¹ represents an optionally substituted straight-chained, branched or cyclic alkyl group; and
each R² independently represents a hydrogen atom or an optionally substituted alkyl group, or a pharmaceutically acceptable salt thereof.

63. The method of claim 62, wherein in each R² the optionally substituted alkyl group is an R¹ group.

64. The method of claim 62, wherein in formula I, R¹ either represents an unsubstituted alkyl group or an alkyl group substituted by an aryl group and/or each R² represents a hydrogen atom.

65. The method of claim 62, wherein said compound is selected from the group consisting of 1-methylpentyl ALA ester, p-isopropylbenzyl ALA ester, p-methylbenzyl ALA ester, benzyl ALA ester, 2-phenylethyl ALA ester, hexyl ALA ester, cyclohexyl ALA ester, 4-methylbenzyl ALA ester, p-[tri-fluoromethyl]benzyl ALA ester, p-[t-butyl]benzyl ALA ester, p-nitrobenzyl ALA ester, 1-ethylbutyl ALA ester, 2-methylbenzyl ALA ester, 4-phenyl butyl ALA ester, p-fluorobenzyl ALA ester, 3,3-dimethyl-1-butyl ALA ester, 2-fluorobenzyl ALA ester, 2,3,4,5,6-pentafluorobenzyl ALA ester, 4-chlorobenzyl ALA ester, 2-methoxyethyl ALA ester, 3-nitrobenzyl ALA ester, 3,4-[di-chloro]benzyl ALA ester, 3,6-dioxa-1-octyl ALA ester, 3-fluorobenzyl ALA ester, 3,6,9-trioxa-1-decyl ALA ester, 3-pyridinyl-methyl ALA ester, 4-diphenyl-methyl ALA ester, 4-methoxy-benzyl ALA ester, 2-methylbenzyl ALA ester, benzyl-5-[(1-acetyloxyethoxy)-carbonyl]amino levulinate, and 3-methylbenzyl ALA ester, and pharmaceutically acceptable salts thereof.

66. The method of claim 61, wherein said first photosensitizer is 5-ALA methyl ester, 5-ALA hexyl ester, 5-ALA benzyl ester, or pharmaceutically acceptable salts thereof.

67. The method of claim 60, wherein said second photosensitizer is a hematoporphyrin, a chlorin, or a sulphonated phthalocyanine.

68. A kit or pack containing a first photosensitizer which is a derivative or analog of 5-aminolevulinic acid (5-ALA), or pharmaceutically acceptable salts thereof, formulated with one or more physiologically acceptable carrier or excipient, and separately a second photosensitizer for simultaneous, separate or sequential use in treating a wound arising from a thermal injury or a wound associated with an ulcer.

69. The kit or pack of claim 68, wherein said first photosensitizer is an ester of 5-ALA or an N-substituted derivative thereof.

70. The kit or pack of claim 69, wherein said first photosensitizer is a compound of general formula I:
R²₂N—CH₂COCH₂—CH₂CO—OR¹ (I)
wherein R¹ represents an optionally substituted straight-chained, branched or cyclic alkyl group; and
each R² independently represents a hydrogen atom or an optionally substituted alkyl group, or a pharmaceutically acceptable salt thereof.

71. The method of claim 70, wherein in each R² the optionally substituted alkyl group is an R¹ group.

72. The method of claim 70, wherein in formula I, R¹ either represents an unsubstituted alkyl group or an alkyl group substituted by an aryl group and/or each R² represents a hydrogen atom.

73. The method of claim 70, wherein said compound is selected from the group consisting of 1-methylpentyl ALA ester, p-isopropylbenzyl ALA ester, p-methylbenzyl ALA ester, benzyl ALA ester, 2-phenylethyl ALA ester, hexyl ALA ester, cyclohexyl ALA ester, 4-methylbenzyl ALA ester, p-[tri-fluoromethyl]benzyl ALA ester, p-[t-butyl]benzyl ALA ester, p-nitrobenzyl ALA ester, 1-ethylbutyl ALA ester, 2-methylbenzyl ALA ester, 4-phenyl butyl ALA ester, p-fluorobenzyl ALA ester, 3,3-dimethyl-1-butyl ALA ester, 2-fluorobenzyl ALA ester, 2,3,4,5,6-pentafluorobenzyl ALA ester, 4-chlorobenzyl ALA ester, 2-methoxyethyl ALA ester, 3-nitrobenzyl ALA ester, 3,4-[di-chloro]benzyl ALA ester, 3,6-dioxa-1-octyl ALA ester, 3-fluorobenzyl ALA ester, 3,6,9-trioxa-1-decyl ALA ester, 3-pyridinyl-methyl ALA ester, 4-diphenyl-methyl ALA ester, 4-methoxy-benzyl ALA ester, 2-methylbenzyl ALA ester, benzyl-5-[(1-acetyloxyethoxy)-carbonyl]amino levulinate, and 3-methylbenzyl ALA ester, and pharmaceutically acceptable salts thereof.

74. The method of claim 69, wherein said first photosensitizer is 5-ALA methyl ester, 5-ALA hexyl ester, 5-ALA benzyl ester, or pharmaceutically acceptable salts thereof.

75. The kit or pack of claim 68, wherein said second photosensitizer is a hematoporphyrin, a chlorin, or a sulphonated phthalocyanine.

76. A product or kit for use in a method of treating a wound arising from a thermal injury or a wound associated with an ulcer comprising:
(a) a first container containing a photosensitizer which is a derivative or analog of 5-aminolevulinic acid (5-ALA), or pharmaceutically acceptable salts thereof, formulated with one or more physiologically acceptable carrier or excipient; and
(b) a second container containing an antiseptic or an antibiotic.

77. A product or kit of claim 76 which further comprises one or more components selected from a second photosensitizer, a surface-penetration assisting agent and a chelating agent.

78. The method of claim 76, wherein said photosensitizer is an ester of 5-ALA or an N-substituted derivative thereof.

79. The method of claim 78, wherein said photosensitizer is a compound of general formula I:
R²₂N—CH₂COCH₂—CH₂CO—OR¹ (I)
wherein R¹ represents an optionally substituted straight-chained, branched or cyclic alkyl group; and
each R² independently represents a hydrogen atom or an optionally substituted alkyl group, or a pharmaceutically acceptable salt thereof.

80. The method of claim 79, wherein in each R² the optionally substituted alkyl group is an R¹ group.

81. The method of claim 79, wherein in formula I, R¹ either represents an unsubstituted alkyl group or an alkyl group substituted by an aryl group and/or each R² represents a hydrogen atom.

82. The method of claim 79, wherein said compound is selected from the group consisting of 1-methylpentyl ALA ester, p-isopropylbenzyl ALA ester, p-methylbenzyl ALA ester, benzyl ALA ester, 2-phenylethyl ALA ester, hexyl ALA ester, cyclohexyl ALA ester, 4-methylbenzyl ALA ester, p-[tri-fluoromethyl]benzyl ALA ester, p-[t-butyl]benzyl ALA ester, p-nitrobenzyl ALA ester, 1-ethylbutyl ALA ester, 2-methylbenzyl ALA ester, 4-phenyl butyl ALA ester, p-fluorobenzyl ALA ester, 3,3-dimethyl-1-butyl ALA ester, 2-fluorobenzyl ALA ester, 2,3,4,5,6-pentafluorobenzyl ALA ester, 4-chlorobenzyl ALA ester, 2-methoxyethyl ALA ester, 3-nitrobenzyl ALA ester, 3,4-[di-chloro]benzyl ALA ester, 3,6-dioxa-1-octyl ALA ester, 3-fluorobenzyl ALA ester, 3,6,9-trioxa-1-decyl ALA ester, 3-pyridinyl-methyl ALA ester, 4-diphenyl-methyl ALA ester, 4-methoxy-benzyl ALA ester, 2-methylbenzyl ALA ester, benzyl-5-[(1-acetyloxyethoxy)-carbonyl]amino levulinate, and 3-methylbenzyl ALA ester, and pharmaceutically acceptable salts thereof.

83. The method of claim 78, wherein said photosensitizer is 5-ALA methyl ester, 5-ALA hexyl ester, 5-ALA benzyl ester, or pharmaceutically acceptable salts thereof.

84. A method for manufacturing a medicament for use in treating a wound which is substantially free from infection by a pathogenic microorganism, said method comprising formulating a photosensitizer which is a derivative or analog of 5-aminolevulinic acid (5-ALA), or pharmaceutically acceptable salts thereof, with one or more physiologically acceptable carrier or excipient.

85. The method of claim 84, wherein said photosensitizer is an ester of 5-ALA or an N-substituted derivative thereof.

86. The method of claim 85, wherein said photosensitizer is a compound of general formula I:
R²₂N—CH₂COCH₂—CH₂CO—OR¹ (I)
wherein R¹ represents an optionally substituted straight-chained, branched or cyclic alkyl group; and
each R² independently represents a hydrogen atom or an optionally substituted alkyl group, or a pharmaceutically acceptable salt thereof.

87. The method of claim 85, wherein in each R² the optionally substituted alkyl group is an R¹ group.

88. The method of claim 85, wherein in formula I, R¹ either represents an unsubstituted alkyl group or an alkyl group substituted by an aryl group and/or each R² represents a hydrogen atom.

89. The method of claim 85, wherein said compound is selected from the group consisting of 1-methylpentyl ALA ester, p-isopropylbenzyl ALA ester, p-methylbenzyl ALA ester, benzyl ALA ester, 2-phenylethyl ALA ester, hexyl ALA ester, cyclohexyl ALA ester, 4-methylbenzyl ALA ester, p-[tri-fluoromethyl]benzyl ALA ester, p-[t-butyl]benzyl ALA ester, p-nitrobenzyl ALA ester, 1-ethylbutyl ALA ester, 2-methylbenzyl ALA ester, 4-phenyl butyl ALA ester, p-fluorobenzyl ALA ester, 3,3-dimethyl-1-butyl ALA ester, 2-fluorobenzyl ALA ester, 2,3,4,5,6-pentafluorobenzyl ALA ester, 4-chlorobenzyl ALA ester, 2-methoxyethyl ALA ester, 3-nitrobenzyl ALA ester, 3,4-[di-chloro]benzyl ALA ester, 3,6-dioxa-1-octyl ALA ester, 3-fluorobenzyl ALA ester, 3,6,9-trioxa-1-decyl ALA ester, 3-pyridinyl-methyl ALA ester, 4-diphenyl-methyl ALA ester, 4-methoxy-benzyl ALA ester, 2-methylbenzyl ALA ester, benzyl-5-[(1-acetyloxyethoxy)-carbonyl]amino levulinate, and 3-methylbenzyl ALA ester, and pharmaceutically acceptable salts thereof.

90. The method of claim 84, wherein said photosensitizer is 5-ALA methyl ester, 5-ALA hexyl ester, 5-ALA benzyl ester, or pharmaceutically acceptable salts thereof.
